(19) United States
(12) Patent Application Publication
(10) Pub. No.: US 2023/0225859 A1
(43) Pub. Date: Jul. 20, 2023

(54) INTRAOCULAR LENS DELIVERY SYSTEM

(71) Applicant: Beaver-Visitec International, Inc., Waltham, MA (US)

(72) Inventors: Nathan Stewart Cauldwell, Hope, RI (US); Paula Yowen Wang, Sudbury, MA (US); Alexander Yuping Zhu, Wayland, MA (US)

(73) Assignee: Beaver-Visitec International, Inc., Waltham, MA (US)

(21) Appl. No.: 18/098,665

(22) Filed: Jan. 18, 2023

Related U.S. Application Data
(60) Provisional application No. 63/300,819, filed on Jan. 19, 2022.

(51) Int. Cl. A61F 2/16

(52) U.S. Cl. CPC A61F 2/1678; A61F 2/167

(57) ABSTRACT

A semi-loaded intralocular lens injector device and related methods are described. In some embodiments, an intraocular lens is stored in a non-deformed configuration in a cassette separate from the injector device. When the cassette is inserted into a cavity of the injector device, a projection extending into the cavity deforms the intraocular lens into a predetermined configuration.

### RELATED APPLICATIONS

This application claims the benefit under 35 U.S.C. § 119(e) to U.S. Provisional Application Ser. No. 63/300,819, filed on Jan. 19, 2022, which is hereby incorporated by reference in its entirety.

### FIELD

Disclosed embodiments are related to intraocular lens delivery systems.

### BACKGROUND

Intraocular lens injector devices insert an intraocular lens into an eye to replace a lens removed during a cataract operation. The injector may be pre-loaded with an intraocular lens or a user may manually load a lens into the device prior to injecting into a patient's eye.

### SUMMARY

In some embodiments, an intraocular lens injector device comprises a body including a cavity and a projection extending into an interior of the cavity. The cavity is configured to receive a cassette including an intraocular lens disposed therein, wherein when the cassette is inserted into the cavity, the projection deforms the intraocular lens to a predetermined configuration.

In some embodiments, a method comprises inserting a cassette into a cavity of an intraocular lens injector device, wherein the cassette includes an intraocular lens disposed therein. The method further comprises deforming the intraocular lens to a predetermined configuration with a projection extending into an interior of the cavity as the cassette is inserted into the cavity.

In some embodiments, a cassette for holding an intraocular lens comprises a body configured to be inserted into a cavity of an intraocular lens injector device. The cassette further comprises an opening extending through at least a portion of the body from a first surface of the body to a second surface of the body located opposite from the first surface, wherein the opening is configured to receive the intraocular lens therein. The cassette further comprises one or more ledges configured to support the intraocular lens in the opening of the cassette when the intraocular lens is disposed therein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 depicts one embodiment of an intraocular lens injector;

FIG. 2 depicts one embodiment of a distal portion of an intraocular lens injector in an open configuration and a cassette before the cassette is attached to the injector;

FIGS. 3A-3C depict one embodiment of a cassette;

FIGS. 4A-4C depict one embodiment of a cassette with an intraocular lens disposed therein;

FIG. 5A depicts one embodiment of a cassette attached to a distal portion of an intraocular lens injector in an open configuration;

FIG. 5B depicts the cassette and intraocular lens injector of FIG. 5A in a closed configuration;

FIG. 5C depicts the cassette and intraocular lens injector of FIG. 5A in a closed configuration and with the folding bar removed;

FIG. 6A depicts a cross-sectional schematic of one embodiment of an intraocular lens injector in a closed configuration with an intraocular lens in a folded position;

FIG. 6B depicts the intraocular lens injector of FIG. 6A with a folding bar partially removed;

FIG. 6C depicts the intraocular lens injector of FIG. 6A with a folding bar completely removed;

FIG. 6D depicts an enlarged view of the intraocular lens of FIG. 6C;

FIG. 6E depicts a cross-sectional perspective schematic of one embodiment of an intraocular lens injector in a closed configuration with an intraocular lens in a folded position;

FIG. 7 depicts one embodiment of a distal portion of an intraocular lens injector in a closed configuration;

FIG. 8 depicts one embodiment of a distal portion of an intraocular lens injector with a cover removed;

FIGS. 9A-9B depict one embodiment of a distal portion of a plunger;

FIGS. 10A-10B depict one embodiment of a distal portion of a plunger;

FIG. 11 depicts one embodiment of a distal portion of a plunger;

FIGS. 12A-12B depict one embodiment of an intraocular lens injector and a package containing a cassette;

FIG. 13 depicts one embodiment of a shuttle in an intraocular lens injector;

FIG. 14 depicts one embodiment of a cassette and a distal portion of an intraocular lens injector;

FIGS. 15A-15B depict one embodiment of a cassette and a cassette package;

FIGS. 16A-16C depict one embodiment of a distal portion of an intraocular lens injector;

FIG. 17A depicts one embodiment of an intraocular lens injector;

FIG. 17B depicts a cross-sectional perspective schematic of a distal portion of the intraocular lens injector of FIG. 17A;

FIG. 17C depicts a perspective view of a distal portion of the intraocular lens injector of FIG. 17A;

FIG. 18A depicts a cross-sectional perspective schematic of one embodiment of an intraocular lens injector in a first position; and

FIG. 18B depicts a cross-sectional perspective schematic of the intraocular lens injector a FIG. 18A in a second position.

### DETAILED DESCRIPTION

Cataract surgery involves replacing a natural lens of the eye that has developed an opacification or cataract with an intraocular lens (“IOL”). A doctor may insert the IOL manually through an incision in the eye. Alternatively, a doctor may use an injector device, which may involve pushing an IOL in a deformed configuration (e.g., folded or rolled up) through a narrow opening of the injector device and into a patient's eye. After insertion, the IOL may unfold, and a surgeon may properly position the IOL in the patient's eye. Because the IOL may be inserted in the eye in a deformed configuration, a smaller incision may be used than with manual insertion which requires much larger incision.

An IOL injector device may be unloaded or preloaded with an IOL. In unloaded devices, a doctor may manually load an IOL into the injector using forceps or other medical tools prior to a cataract procedure. Because a doctor handles the IOL, manual loading increases the risk of contamination and user error. Preloaded injector devices may eliminate any need to touch the IOL and therefore reduce these risks, but because the IOL is already loaded in the device, it cannot be stored in a folded configuration for long durations of time due to risk of permanently deforming the IOL.

In view of the above, the inventors have recognized and appreciated designs for a semi-loaded IOL delivery system that stores an IOL in an undeformed configuration while also reducing the risk of contamination and user error. In some embodiments, the system includes a cassette separate from the injector device that holds an IOL in a substantially undeformed configuration (e.g., flat configuration). Prior to a cataract procedure, the cassette may be coupled to the injector device to load the IOL into the injector device. The IOL may be deformed into a curved configuration as the IOL is loaded into the device prior to insertion.

The above embodiment of an IOL cassette and injector may offer a number of benefits. Specifically, in such an embodiment, the IOL may be held in a deformed configuration for a relatively short duration of time prior to the cataract procedure, reducing or eliminating the risk of permanently deforming the IOL due to being held in a deformed configuration for prolonged time periods. In addition, the cassettes may have a smaller footprint and may be stored separately from an injector device in smaller packages, eliminating bulky storage issues with preloaded devices. Moreover, the cassette may be removed from the sterile package and directly coupled to the injector device without a user handling the IOL, reducing the risk of contamination. In addition, because the injector device deforms the IOL into a curved configuration during insertion into the device, there may be no need for a user to position the IOL, further reducing risk of user error and contamination. Of course, while several advantages are noted above, it should be understood that other advantages may also be realized using the disclosed methods and systems as the current disclosure is not limited in this fashion.

In some embodiments, a cassette loaded with an undeformed IOL may be inserted into a cavity of an injector device. The cavity may be positioned at a proximal end portion of the injector device and operatively connected to a nozzle on the distal end of the device. The cavity may include a projection that extends into an interior volume of the cavity. When the cassette is inserted into the cavity, the projection may deform the IOL into a predetermined configuration, such as a curved shape. In some embodiments, after the IOL is deformed, the projection may be removed at least partially from the cavity out of a path of a plunger positioned proximally relative to the IOL within the cavity prior to deployment. The plunger may move in a distal direction to push the deformed IOL through the cavity and the nozzle. As the IOL moves distally through the narrowing nozzle, the IOL may continue to deform into a tighter, curved configuration to fit through an opening in a distal end portion of the nozzle.

In some embodiments, an injector device may include a cover that is moveably coupled to a body of the injector device. The cover may move between an open configuration and a closed configuration. In the closed configuration, the cover may be positioned over a cavity disposed in the injector device. In some embodiments, a cassette loaded with an undeformed IOL may be coupled to a surface of the cover when the cover is in the open configuration. Moving the cover from the open configuration to the closed configuration may insert the cassette into the cavity to load the IOL into the cavity. In some embodiments, the cover may be rotationally coupled to the body via a hinge such that the cover moves between the open and closed configurations in a rotational movement. During loading (i.e., rotating from the open to closed configuration), a projection extending into the cavity may deform the IOL into a curved shape as the cover, and the coupled cassette and IOL, are displaced into the cavity and into engagement with the projection. As such, the IOL is deformed as the injector device is loaded to prepare for deploying the IOL during a procedure.

As noted above, in some embodiments, a cassette including an undeformed IOL may be inserted into a cavity of an injector device to load the IOL into the injector device. Various features of the injector device and cassette, including the exemplary projection extending into the cavity described above, may deform the IOL as it is loaded into the cavity. In some embodiments, the cassette may include an opening that extends from a first surface to a second opposing surface of the cassette, though embodiments in which the opening does not extend all the way through the cassette are also contemplated. The IOL may be supported within a portion of the opening by one or more appropriately configured ledges. For example, the ledges may be sized and shaped to both support and retain one or more non-optical portions of the IOL to retain the IOL within the opening as elaborated on below. The cavity may include a projection that extends at least partially into, and in some embodiments, through the opening of the cassette as the cassette is inserted into the cavity to deform the IOL within the opening. In some embodiments, the second surface, or other appropriate portion, of the cassette may be disposed proximate to a portion of the injector device that has a curved surface positioned adjacent to the opening of the cassette when the cassette is received in the cavity and the device is in the closed configuration. Thus, when the projection extends through the opening of the cassette, the projection may press against a center portion of the IOL while outer portions of the IOL are retained by the ledges in the opening, causing the IOL to deform toward and/or against the curved surface to provide a desired initial curved shape of the IOL.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

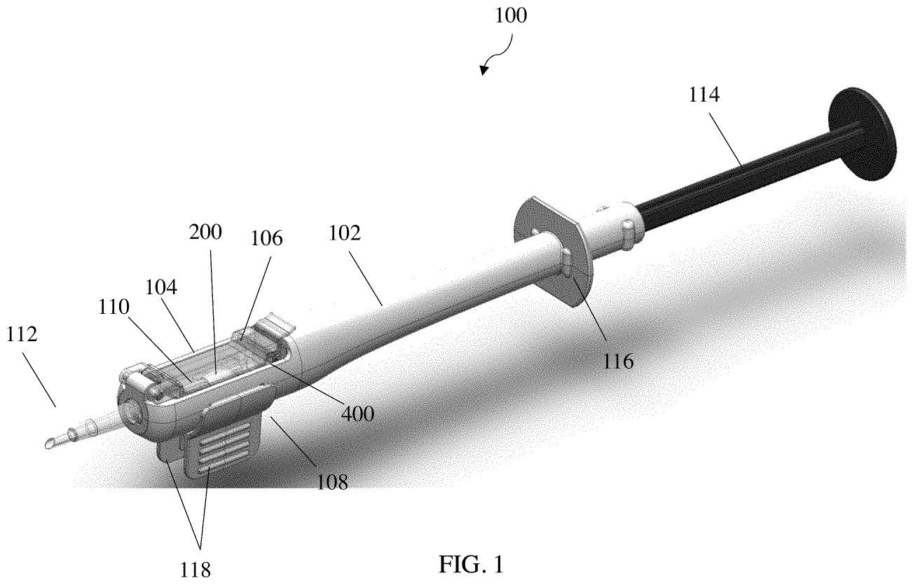

FIG. 1 shows an intraocular lens (IOL) injector device 100 according to one embodiment. The IOL injector device 100 includes an elongated body 102 having a cavity 104 formed in a distal portion of the body and a cover 106 moveably attached to the body 102 at a location proximate to the cavity. As shown in FIG. 1, the cover 106 is disposed over the cavity opening when in a closed configuration. A shuttle 108 is configured to be selectively moved relative to the body 102 to control a position of a projection 110 operatively coupled to the shuttle and extending at least partially into the cavity 104 when the shuttle 108 is engaged with the body 102. As shown in FIG. 1, an IOL 200 is disposed in the cavity 104 and held in a deformed configuration between the cover 106 and the projection 110 when the device is in the closed configuration. Additionally, in some embodiments, the shuttle and associated projection may be removed from the cavity and body, or at least moved out of engagement with the IOL, after deforming the IOL to a desired configuration.

As shown in FIG. 1, in this embodiment, the injector device 100 includes a nozzle 112 that extends distally from a distal end portion of the body 102. The nozzle 112 is fluidly connected to the cavity 104. An actuation device such as a piston 114 is disposed within and extends proximally from a proximal end portion of the body 102. The piston 114 is operatively coupled to a plunger 400 disposed proximally from a portion of the cavity 104 including the IOL in the loaded and closed configuration. The piston 114 and plunger 400 may be axially aligned with the nozzle 112 in some embodiments with the cavity 104 and deformed IOL 200 disposed therebetween when the device is in the loaded and closed configuration. Accordingly, actuating the piston 114 (i.e., compressing the piston 114 in a distal direction along a longitudinal axis of the injector device 100) may move the plunger 400 in a distal direction through the cavity 104 to push the IOL 200 200 through the cavity 104 and the nozzle 112 and out a distal opening in the nozzle 112. It should be noted that although a piston 114 is shown and described, the injector device may include other deployment systems (i.e., direct plunge, screw design) to distally displace the plunger and deploy the IOL out of the device.

In some embodiments, a diameter, or other transverse dimension, of an internal channel extending through the nozzle 112 decreases in a distal direction. Thus, as the IOL is distally displaced through the nozzle 112, the IOL 200 continues to deform and roll into a smaller configuration to fit through the nozzle 112. When the IOL is disposed at the distal opening of the nozzle 112, the IOL may be in a second deformed configuration that has a transverse dimension (e.g., a diameter) that is smaller than the initial deformed configuration of the IOL upon loading into the device. As such, the IOL may be inserted through a small incision in the patient's eye via the nozzle 112 inserted into the incision. Once the IOL 200 is inserted into the eye, the IOL may unfold into a non-deformed configuration and the surgeon may properly position the IOL in the eye.

In some instances it may be desirable to provide one or more features to facilitate grasping and/or other manual manipulation of a device. For example, as shown in FIG. 1, the injector device 100 may include a flange 116 (e.g., a barrel flange or other radially extending structure) that extends radially from a proximal portion of the body 102. A surgeon may grip the flange 116 and the piston 114 with one hand to actuate the piston 114. In some embodiments, the flange 116 may have a flat surface bottom and/or top surface to prevent the injector device 100 from tipping over when placed on another surface (e.g., table or counter). In some embodiments, the shuttle 108 may include two parallel legs 118 that extend away from a bottom surface of the body 102 to help support and stabilize the injector device 100 on a supporting surface the injector device is disposed on. However, the legs, a flat base, and/or any other appropriate structure configured to maintain a desired orientation of the injector device on a supporting surface may be used as the disclosure is not so limited.

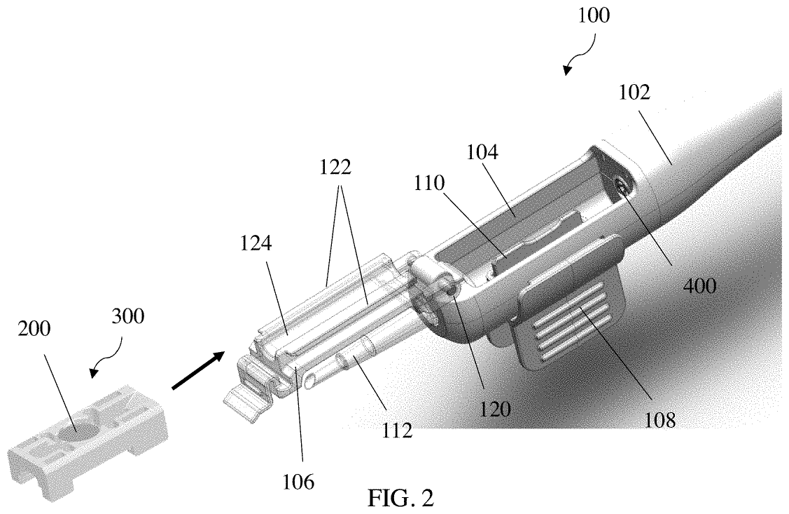

FIG. 2 shows one embodiment of a distal portion of an injector device 100 with a cover 106 moveably attached to a body 102 in an open configuration. The cover 106 may be rotatably attached to the body via a hinge 120 and the cover 106 may rotate between an open configuration (FIG. 2) and a closed configuration (FIG. 1). Of course, the cover 106 may be attached to the body 102 using other attachment features and the cover may move relative to the body in other directions, as the disclosure is not so limited. For example, embodiments in which a cassette is insertable into the cavity 104 in a direction that is transverse to a longitudinal axis of the injector device without the aid of a hinge are also contemplated. As shown in FIG. 2, in some embodiments the injector device 100 is not preloaded with an IOL 200. In some embodiments, an IOL 200 may be stored in a cassette 300 that is configured to attach to the injector device 100 to load the IOL 200 into the injector device 100. The IOL 200 may be stored and held in the cassette 300 in a substantially non-deformed configuration, and the cassette 300 and IOL 200 may be stored together in a sterile package. In a non-limiting example, a surgeon may open a sterile package containing a cassette 300 and a non-deformed IOL 200 and attach the cassette 300 directly to an injector device 100 by sliding the cassette onto a surface of a cover 106, or otherwise connecting the cassette to a desired portion of the injector device, when the cover is in an open configuration. The cassette 300 may be attached to the cover 106 by various types of connectors. For example, this may include sliding the cassette 300 in a direction oriented along a length of the cover, as indicated by the arrow shown in FIG. 2, into engagement with one or more rails 122 on an interior surface of the cover 106. It should be noted that other types of connections for attaching the cassette to the cover, or another portion of the injector device, are also contemplated (e.g., a snap fit, magnetic connectors, mechanically interlocking features, threaded fasteners, and/or any other appropriate type of connector), as the disclosure is not so limited.

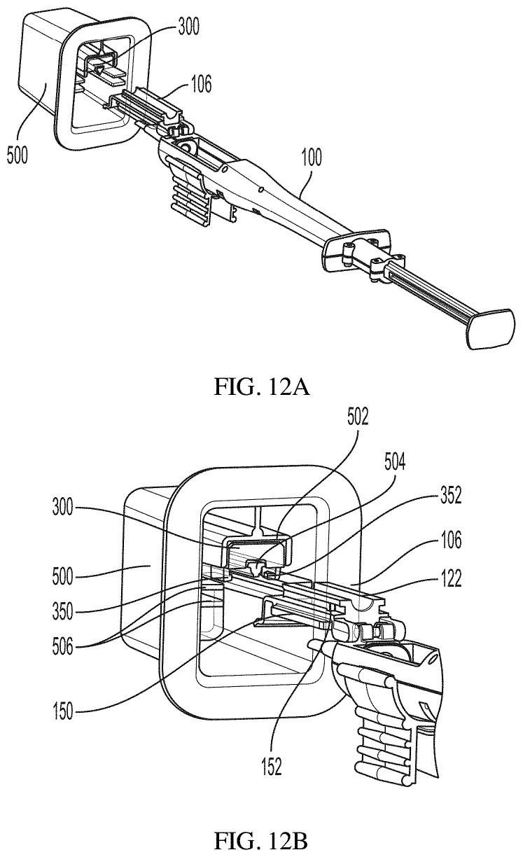

In some embodiments, a surgeon or other user may attach a cassette to the injector device without directly handling the cassette. For example, as shown in FIGS. 12A-12B, a surgeon may insert a cover 106, or other corresponding portion, of the injector device 100 into a sterile package 500 containing a cassette 300. The cassette 300 may be held within the package 500 in a direction that is approximately perpendicular to an opening of the package by projecting walls 502 and rod 504. The opening of the package 500 may include two pairs of parallel ledges 506 on opposite walls to receive outside edges 150 of the cover 106. The ledges 506 may properly align the cover 106 with the cassette 200 as the cover is inserted into the opening of the package in a perpendicular direction. As the surgeon inserts the cover 106 into the opening of the package 500, the cover 106 may slide into engagement with the cassette 200. In some embodiments, one or more ledges 350, or other retention structures, of the cassette may slide over the rails 122 of the cover to attach the cassette to the cover. In some embodiments, the cover may include detents 152 that slide past ribs 352 in the ledges 350 or other portion of the cassette to attach the cassette to the cover such that the cassette is retained on the cover when the surgeon removes the cover from the package 500. Use of the attached cassette and device may then proceed as discussed elsewhere herein.

It should be noted that, in some embodiments, a cassette 300 and IOL 200 need not be used with the above-described injector device 100. For example, an IOL 200 stored in cassette 300 may be used in any injector device, as the disclosure is not so limited. In some embodiments, if a cassette 300 is stored in a sterile package 500, a user may remove the cassette 300 from the package 500. A user may then remove the IOL 200 directly from the cassette 300; e.g., by using forceps to lift the IOL out of the cassette without damaging the IOL 200, prior to positioning the IOL in a separate injector device for subsequent implantation. Accordingly, the cassette 200 may store the IOL 200 prior to the IOL 200 being loaded into any injector device as the disclosure is not limited in this fashion.

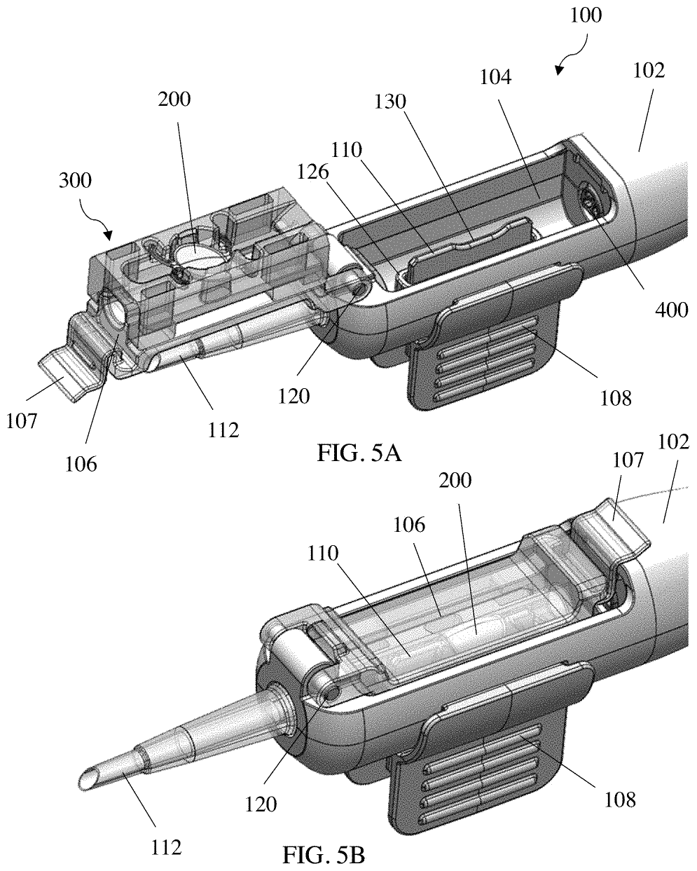

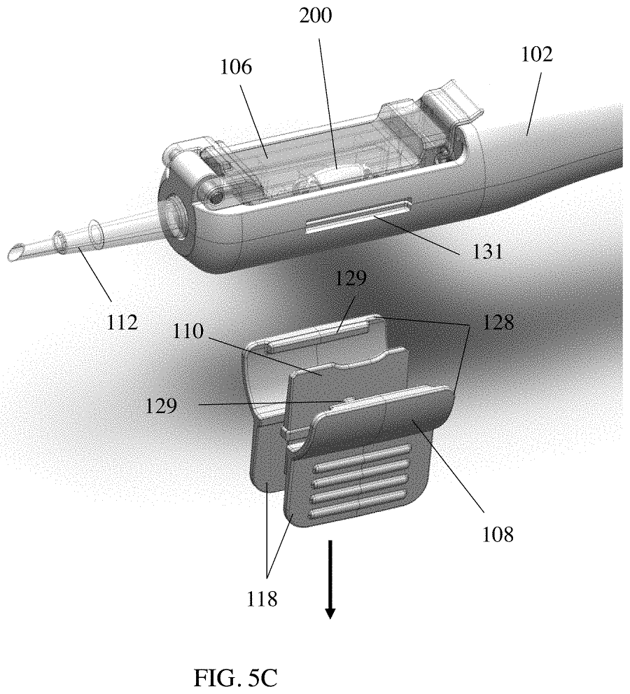

As discussed in more detail below with respect to FIGS. 5A-5B, once the cassette 300 is connected to the cover 106, the cover 106 may then be moved from the open configuration to a closed configuration (e.g., by rotation of the cover). Regardless of how the cover is closed, as the cover is closed, the cassette 300 and IOL 200 may be inserted into the cavity 104 as the cover 106 is moved towards the closed configuration. As the IOL 200 enters the cavity 104, a projection 110 extending into the cavity may contact and press the IOL into a curved surface 124 formed on the cover 106 and that is oriented towards the IOL when the cassette 300 is assembled with the cover 106. Accordingly, the projection may deform the IOL into a curved or folded configuration against the curved surface, where the IOL is positioned in line with a path of travel of a distally moving plunger. As such, the IOL may be stored in a substantially non-deformed configuration in the cassette prior to being deformed as it is inserted into an injector device. This may reduce, or substantially prevent, damage to the IOL 200 from prolonged storage in a deformed configuration. In addition, the surgeon may load the IOL 200 into the injector device without directly handling the IOL 200 which may also reduce a risk of contamination and/or user error associated with manual manipulation of the IOL.

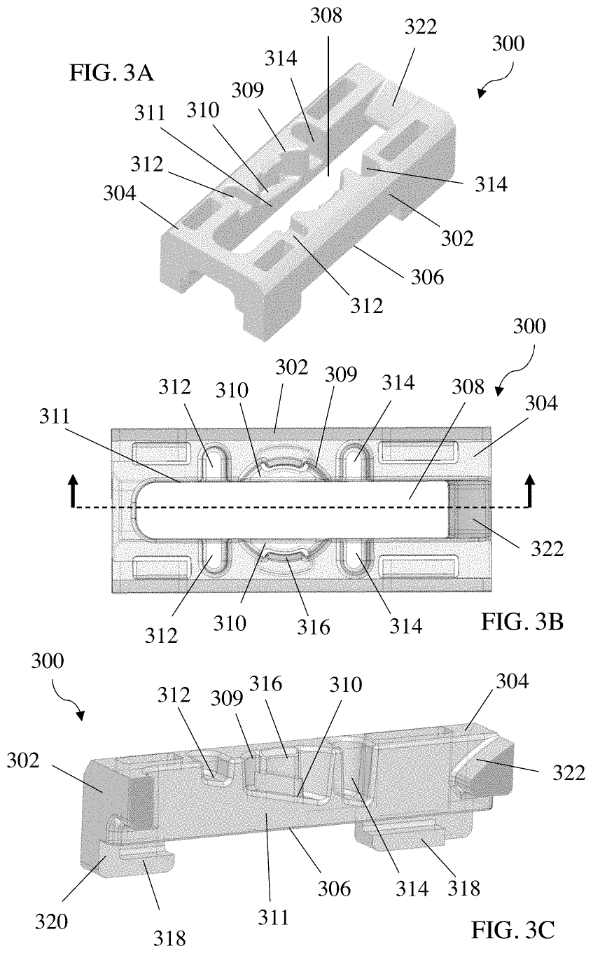

FIGS. 3A-3C show one embodiment of a cassette 300 that may be attached to an injector device to load an IOL into the device. As shown in FIGS. 3A-3C, a cassette 300 includes a body 302 having an opening 308 that extends through at least a portion of the body 302 from a first surface 304 to a second surface 306 of the body that may be opposite from the first surface. The opening may be an elongated opening with a maximum dimension of the opening oriented substantially along a length or longitudinal axis of the body. In some instances, the opening may be sized and shaped to accommodate the insertion of a projection from an injector device into the opening as described above in order to deform an IOL disposed in the opening. However, it should be understood that any appropriately shaped opening may be used. The body may include one or more ledges 310 that are configured to support one or more portions of an IOL disposed thereon. Thus, the ledges 310 may support and retain an IOL in the opening 308. For example, in some embodiments, a recess 309 shaped and sized to fit an IOL may extend into the body 302 from the first surface 304 of the body. The recess 309 may have a larger diameter, or other transverse dimension, than a corresponding width of the opening 308. A depth of the recess may extend partially between the first surface 304 and the second surface 306. Thus, the recess may include a pair of symmetrical ledges 310 disposed on opposite sides of the opening 308. Detents 316, which may correspond to protrusions extending radially inwards from the side walls of the recess on either side of the opening 308, may help to retain a portion of an IOL in the recess 309 between the supporting ledge 310 and associated detent 316.

In some instances, it may be desirable to avoid contacting a single portion of an IOL with a protrusion, or other structure, during insertion into an injector device. Accordingly, in instances in which an IOL is inserted into a cavity via a rotational motion, it may be desirable to angle the IOL relative to a surface of the cassette to avoid contacting a single portion of the IOL with a corresponding projection in a cavity during initial insertion into a cavity. Accordingly, in some embodiments, and as best shown in FIG. 3C, the ledges 310, or other supporting surface of the recess 309, may be angled relative to the first surface 304 and/or the second surface 306 of the body such that an IOL may be supported in the cassette on an incline. In some embodiments, a maximum angle of the incline may be greater than or equal to 8°, 10°, 12°, and/or any other appropriate angle. The maximum angle of the incline may also be less than or equal to 18°, 15°, 13°, and/or any other appropriate angle. Combinations of the foregoing are contemplated including, for example, a maximum angle of the incline that is between or equal to 8° and 18°, and/or any other appropriate combination of the foregoing. While specific ranges for the maximum angle of the incline are provided above, it should be understood that ranges both greater than and less than those noted above are also contemplated as the disclosure is not so limited.

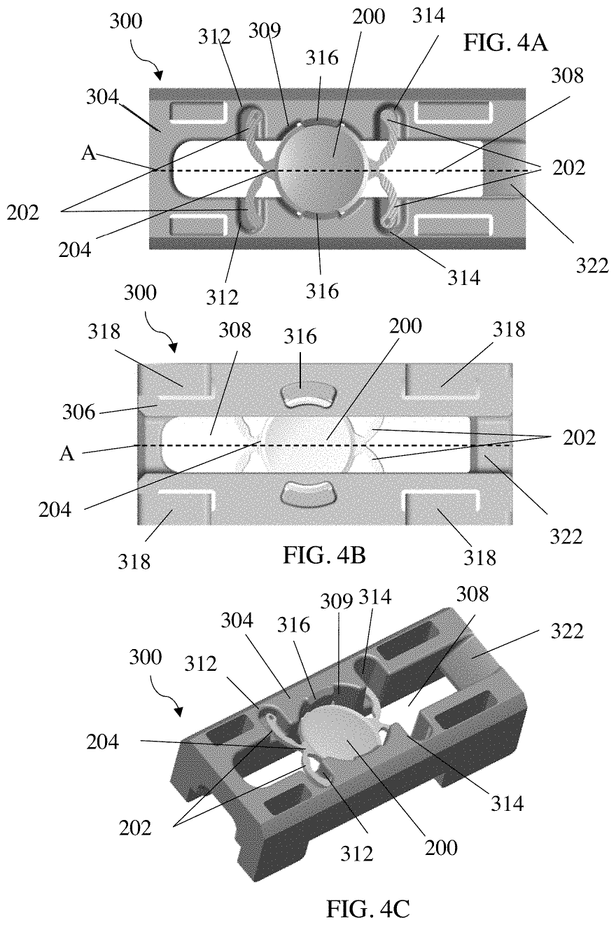

A cassette 300 may also include one or more features to help retain an IOL in a desired position and orientation relative to a recess 309 and associated opening 308 of the cassette. In some such embodiments, the body 302 may include one or more haptic recesses 312 and 314 that extend into walls 311 of the opening. The haptic recesses may be configured to receive one or more haptics 202 of an IOL (e.g., non-optical protrusions connected to a lens of the IOL), see FIGS. 4A-4C. Bottom surfaces of the haptic recesses 312 and 314 oriented outwards towards the first surface may be angled on a similar incline as ledges 310. In some embodiments, the inclined surfaces of the haptic recesses 312 and 314 as well as the inclined surface of the recess may be substantially coplanar with one another. For example, a depth of the haptic recesses 314 may be greater than a depth of the haptic recesses 312 disposed on the other side of the recess in order to help facilitate supporting the IOL in the inclined orientation. In some embodiments, the haptic recesses and ledges 310 may include bumpy surfaces (e.g., bumps, dimples, ribs) to decrease surface contact with the IOL and reduce friction and or stiction during storage.

As shown in FIGS. 3A and 3C, the cassette 300 may include feet 318 that extend from a bottom surface 306 of the body 302. The feet 318 may be configured to slide onto, and be retained by, rails 122 of a cover 106 to attach the cassette to the injector device 100 (see FIG. 2). The cassette may include a mechanical stop 320 at one end of the cassette configured to abut ends of the rails 122 to stop movement of the cassette 300 at a desired position on the cover while preventing the cassette from sliding too far onto the cover 106. The feet 318 and/or the rails 122 may include one or more detents or other physical structure that provides tactile feedback when sliding the cassette onto the rails to indicate the cassette is fully engaged with the injector device.

FIGS. 4A-4C show an IOL 200 disposed in a cassette 300, according to one embodiment. As shown in FIG. 4A, an IOL 200 may be disposed within the recess 309 such that the IOL rests on the ledges formed on opposing portions of the recess on either side of the opening 308. The recess 309 may be customized to fit IOLs of different sizes. Detents 316 gently retain a portion of a perimeter of the IOL 200 surrounding the optical lens to secure the IOL in the recess 309. The detents 316 may be of varying heights to accommodate IOLs of different thicknesses. Haptics 202 extending from the perimeter at vertices 204 are disposed in haptic recesses 312 and 314 of the cassette 200. The haptics may be substantially undeformed within the haptic recesses, though some amount of deformation may be present in the haptics in some embodiments. As shown in FIGS. 4A-4C, the IOL 200 is disposed on an incline within the opening 308 in a non-deformed configuration with an axis extending between the vertices 204 oriented in a direction that is at least partially parallel with a central longitudinal axis of the opening 208.

FIGS. 5A and 5B illustrate the closing of a cover of an injector device as the device transitions between an open and closed configuration. Specifically, FIG. 5A shows a cassette 300 attached to an inside surface of a cover 106 of an injector device 100 in an open configuration. In some embodiments, the cover 106 is moveably attached to the body of the injector device 100 adjacent to a cavity 104. FIG. 5B shows the cover 106 in a closed configuration. In some embodiments, the cover 106 rotates about hinge 120 to position the cover 106 over the cavity 104 and insert a cassette 300 attached to the cover, and IOL 200 retained within the cassette, into the cavity 104. The cassette 300 may be inserted into the cavity up-side down such that the first surface 304 enters the cavity 104 before the second surface 306. In such an embodiment, the first surface and the IOL retained in the cassette may be oriented towards the cavity as the cassette and IOL are inserted into the cavity.

The cover 106 may be moved manually, i.e., such as by grasping handle 107 and rotating the cover until handle 107, or other portion of the cover, is selectively connected to a portion of the body 102 or cavity 104. For example, the handle may have a detent configured to mate with a recess in a cavity wall to secure the cover in the closed configuration though any appropriate type of selectively openable connection may be used. Alternately, the injector device 100 may include a button or other actuation feature that, when pressed, causes the cover to move to a closed configuration. In a non-limiting example, the cover 106 may be spring-loaded such that the cover is biased towards the closed configuration when actuated.

As shown in FIG. 5A, a projection 110 of a shuttle 108 extends at least partially into the cavity 104 through an opening in a bottom portion of the body 102 disposed opposite from the opening of the cavity the cassette 300 is inserted into. A guide wall 126 may at least partially surround the opening through which the projection 110 extends. The guide wall 126 may help to position and support the projection 110 within the cavity 104. In some embodiments, as the cover 106 rotates or otherwise moves to the closed configuration, the IOL 200 is pressed against the projection 110 as the projection 110 is displaced into the opening 208 of the cassette 300. While motion of the cassette 300 and IOL 200 relative to the projection 110 is illustrated in the figure, in some embodiments, the projection 110 may be moved instead to provide the relative motion of these components. In either case, the projection 110 may compress the IOL along axis A (FIGS. 4A-4B) in an upward direction oriented away from the projection through the opening 208. As the IOL 200 is deformed upwards through the opening 208, the ledges 310 (FIGS. 3A-3C) and haptic recesses 312 and 314 may aid in retaining the portions of the IOL and the end portions of the haptics disposed on opposite sides of the opening at an initial height relative to the opening 208. Thus, as a central portion of the IOL 200 is deformed through the opening, this causes the IOL 200 to fold upwards along walls 311 of the cassette 300 and toward and/or against a curved surface 124 of the cover 106 (FIG. 2). Thus, the IOL deforms into a curved shape as the cover (with an attached cassette) of the injector device moves to the closed configuration, which may minimize the time the IOL is in a deformed configuration.

In some embodiments, an IOL may be thicker in a center portion and thinner around a perimeter. The thicker center section may be an optical portion of the IOL that is more susceptible to damage and therefore it may be desirable to minimize, or eliminate, contact with this optical portion of the IOL. In some embodiments, an upper portion of a projection 110 that contacts one or more portions of an IOL may include compliant material which may help to minimize damage to the IOL during deformation. In some embodiments, as shown in FIG. 5A, the projection 110 may also include a cutout 130 that extends at least partially along a length of an upper portion of the projection that contacts the IOL. The cutout 130 may be sized and shaped to avoid contact with one or more portions of the IOL. For example, the cutout 130 may be arranged such that when the cover 106 is moved to the closed configuration over cavity 104 (i.e., by rotating about hinge 120), the cutout 130 is positioned under an IOL 200 such that the thicker center portion of the IOL, corresponding to the optical portion of the IOL, is spaced from (i.e., does not contact) the projection along a length of the cutout. IOLs having greater thicknesses may require a deeper cutout than IOLs having narrower thicknesses to prevent the thicker center portion of the IOL from contacting the projection. Because of the cutout 130, the projection 110 may only contact the IOL at the vertices 204 of the IOL (FIGS. 4A-4C) and/or at other non-optical portions of the IOL. Again, such an arrangement may help to minimize a risk of damage to the optical portion of the IOL. In some embodiments, it may be desirable to provide projections with different sized cut outs for different sized IOL. In such an embodiment, a shuttle 108 may be interchangeable and selected with an appropriately sized cutout 130 on the projection 110 to complement a thickness of a desired IOL.

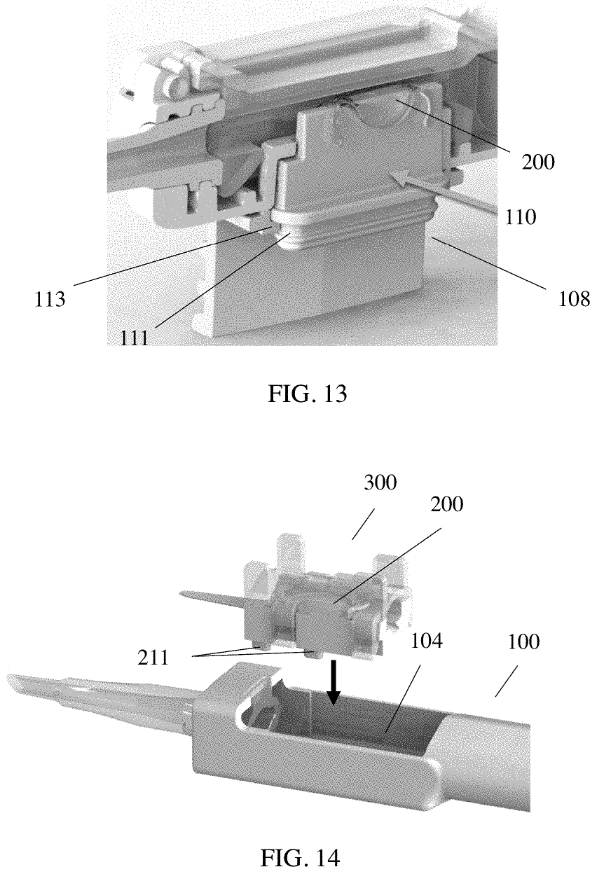

In some embodiments, the projection 110 may be formed separately from the shuttle 108 (see FIG. 13). For example, the projection 110 may be made of a soft material (e.g., silicone) to minimize risk of damage to the IOL 200 when the projection 110 contacts and deforms the IOL 200. The projection 110 may include an attachment end portion 111 configured to attach to the shuttle 108. For example, the attachment end portion 111 may include deformable ribs configured to be inserted into and retained by a protruding ring 113 in an opening of the shuttle. A silicone projection may be made of various sizes and inserted into a shuttle depending on the size IOL used.

As noted previously, in some embodiments, the rotational motion of a cover 106 when moving from the open configuration to the closed configuration causes the cassette 300 to enter the cavity 104 at an angle relative to the projection 110. For example, the end portion of the cassette 300 closest to the hinge 120 enters the cavity 104 before the end portion farthest from the hinge. To prevent the IOL from contacting the projection 110 at an angle, ledges 310 and haptic recesses 312 and 314 hold the IOL in the cassette 300 at an incline relative to the cassette 300 as previously described. The incline is arranged such that, even when the cassette 300 enters the cavity at an angle, the IOL 200 may be positioned substantially parallel to a portion of the projection 110 the IOL contacts during insertion. This may allow the projection 110 to press against both vertices 204, or other portions of the IOL, disposed along a length of the IOL at the same time while avoiding excessive deformation of the IOL at a single location during initial insertion into the cavity. Thus, this arrangement may lead to more uniform folding of the IOL during insertion.

When the cover 106 is completely closed, the projection 110 has deformed the IOL 200 into a curved configuration with the IOL held between walls 311 of the cassette 300 and curved surface 124 of the cover. The projection 110 has pressed the IOL up into the curved surface 124 such that the IOL is no longer inclined and is positioned parallel to the projection 110 and cover 106. In some embodiments, the cover 106 may include a door (not shown) that a surgeon may open to reposition the IOL 200 within the cavity 104 in the event IOL does not fold properly between the projection 110 and the cover 106.

As shown in FIG. 5C, after the IOL 200 is properly positioned in the cavity 104 in a deformed position, the shuttle 108 may be partially or completely removed from the body 102 to move the projection 110 out of contact with the IOL 200 and a path of travel of the IOL through the cavity 104 during deployment. In some embodiments, the shuttle 108 includes one or more connectors 128 that selectively maintain an initial position of the shuttle on the body 102 shuttle prior to partially or completely moving the shuttle and associated projection outwards away from the cavity. In some embodiments, the connection is a latch connection such that the shuttle 108 may be removed by compressing legs 118 together to cause the connectors 128 to pivot outwards and detach from an exterior side of the body 102. In some embodiments, the connectors 128 may include latches 129 on an interior portion that attach to ridges 131 on the outer surface of the body. The body 102 may include more than one ridge 131 along the exterior side of the body to attach the shuttle to the body in more than one position. It should be understood that while a particular type of connector for holding the shuttle in a desired initial position is depicted in the figure, any appropriate type of selective connection (e.g., latches, magnets, temporary adhesives, threaded fasteners, etc.) that permits the shuttle to be moved relative to, or removed from, the body of the injection device may be used as the disclosure is not so limited.

In some embodiments, the shuttle may be pivotally coupled to the injector device. In a non-limiting example, the shuttle may include an elongated arm rotatably attached to the injector device at a first end adjacent a cavity of the injector device. The elongated arm may include a projection at a second end arranged to at least partially extend into an interior of the cavity. The injector device may include a locking device or other securing means to secure the elongated arm in a position to maintain the projection in the cavity. When a cassette including an intraocular lens is disposed in the cavity (e.g., from a side of the injection opposite the elongated arm), the projection may contact and deform the lens in the cassette. Once the lens is in a deformed configuration, a release button on the locking device may release the elongated arm and the projection from the cavity. A spring may bias the arm away from the injector device to remove the projection from the cavity.

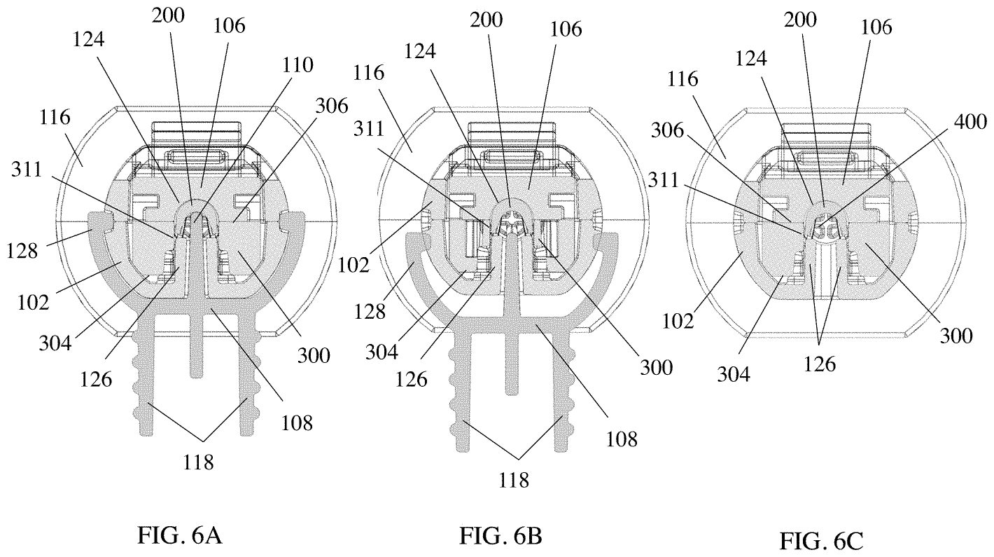

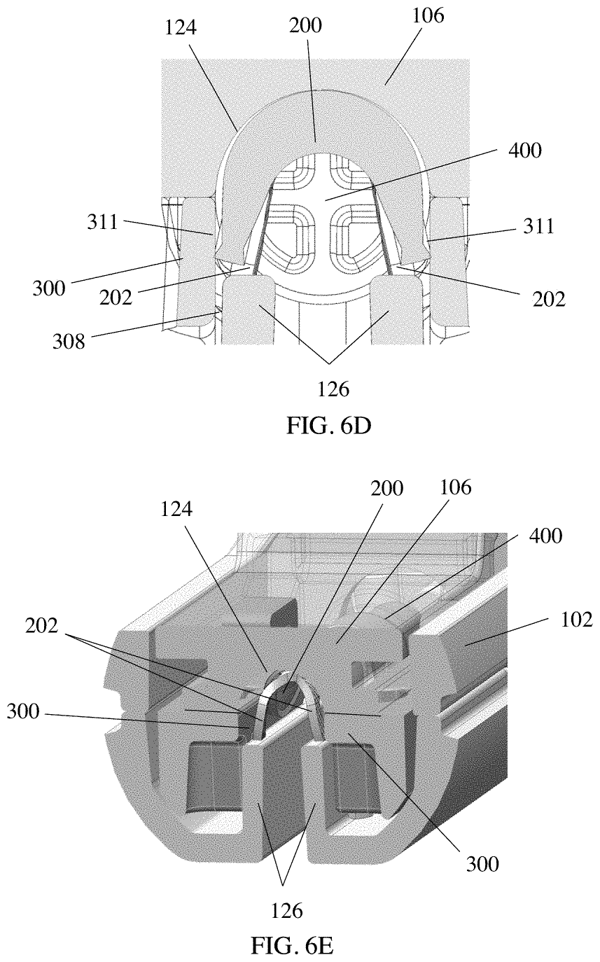

FIGS. 6A-6E are transverse cross-sectional schematics of the injector device taken through the cavity holding an IOL 200 in a deformed configuration and a projection 110 in various positions. In FIG. 6A, the projection 110 extends into the cavity 104 and has pressed the IOL 200 upwards through the opening 308 in the cassette 300 and against the curved surface 124 of the cover 106. The projection 110 extends through an opening in the bottom of the cavity and is disposed between wall 126 which helps stabilize the projection. The IOL 200 is held in the deformed configuration by compressing against wall 311 of the cassette 300 and curved surface 124 of the cover 106. In some embodiments, curved surface 124 may be shaped and positioned a certain distance from the opening such that when the cover is moved to the closed configuration, the projection 110 may press the IOL 200 upwards, deforming the IOL towards the curved surface 124 without pressing the IOL against the curved surface 124. In such embodiments, the IOL may be at least initially held in the deformed configuration by portions of the IOL pressing against walls 311. In some embodiments, the curved surface 124 may form a channel with the inner walls 311 of the cassette 300 that is sized and shaped to fit a plunger 400 configured to push the IOL through the cavity 104 toward a nozzle 112, as described in more detail below. The curved surface 124 may help maintain the IOL in the deformed configuration and guide the IOL as the plunger pushes the IOL through the cavity.

After the IOL 200 is deformed and properly positioned in the cavity 104, the shuttle 108 may be removed. FIG. 6B shows the shuttle 108 partially removed such that the projection is level with an upper surface of wall 126. FIGS. 6C-6D show the shuttle 108 and projection 110 completely removed from the injector device 100. In both positions, the projection 110 has been removed from a path of travel of the IOL 200 and an associated a plunger 400 positioned at a proximal end portion of the cavity 104 where the plunger 400 is configured to move in a proximal direction along a longitudinal axis of the cavity to displace the IOL 200.

FIG. 6D shows an enlarged view of the IOL 200 held in a deformed configuration in the injector device, according to one embodiment. As shown, a perimeter of the IOL 200 contacts an inner wall 311 of the opening 308 extending through cassette. The IOL curves upwards through the opening 308 of the cassette and into the curved surface 124 of the cover. In some embodiments, only the perimeter of the IOL contacts the wall 311, minimizing contact and risk of damage to the optical center portion of the IOL. The walls 311 compress against the perimeter of the IOL and maintain the IOL in the curved configuration, even when the projection 110 is removed. In some embodiments, the upper surface of the guide wall 126 the projection is inserted through may extend into the cavity by an appropriate distance while providing clearance for the IOL perimeter and haptics 202 to be held in the deformed configuration. For example, as best shown in FIG. 6E, the haptics 202 may be disposed above or against the upper surface of wall 126 which may have an appropriate thickness to prevent the haptics 202 from falling into the opening when the projection 110 is removed.

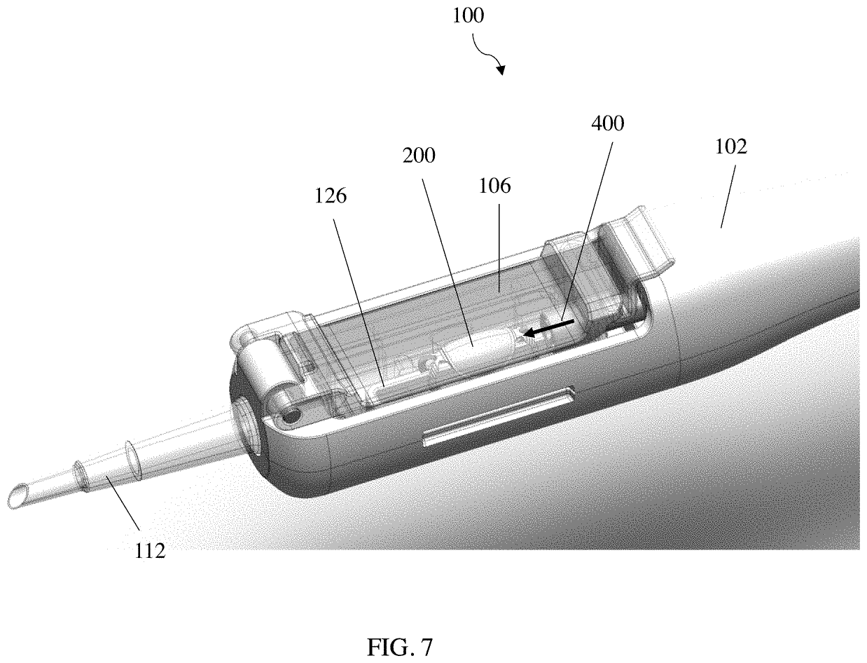

As shown in FIG. 7, a plunger 400 may be actuated to move in a distal direction (shown by the arrow in FIG. 7) through the cavity 104 and nozzle 112 along an axis that is aligned with the IOL 200 when it is in the deformed configuration after the injection device is closed. In some embodiments, as the plunger 400 moves, it pushes the deformed IOL 200 through the cavity 104 and nozzle 112, causing the IOL 200 to roll or curve into a smaller deformed configuration as it travels through the narrowing nozzle before the IOL 200 is pushed out a small distal opening of the nozzle. The IOL 200 may be inserted through a small incision in the eye prior to the IOL unfolding into a nondeformed configuration to be properly positioned in the eye during a cataract procedure. Surfaces of the injector device, including but not limited to curved surface 124, inner walls 311, upper surfaces of wall 126, and inner surface of nozzle 112, may be coated in a lubricant (e.g., low-friction hydrophilic coating) to reduce friction and prevent the IOL 200 from sticking as it travels through the cavity 104 and/or nozzle 112.

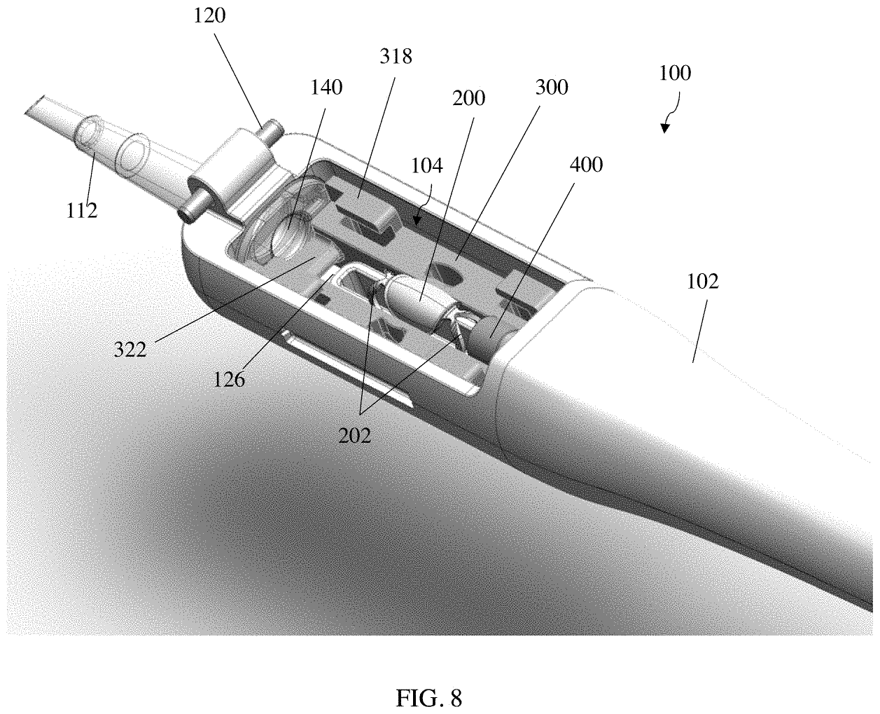

FIG. 8 shows a top perspective view of an injector device 100 according to one embodiment with a cover to cavity 104 removed for visualization purposes. As shown in FIG. 8, a plunger 400 may push an IOL 200 through the cavity 104 into a proximal opening 140 of the cavity connected to nozzle 112 and out a distal end of the nozzle. In some embodiments, larger IOLs may benefit from elongating the cavity 104 downwards to accommodate the larger diameter IOL and attached haptics 202. However, this may cause the IOL 200 to sit lower in the cavity 104 relative to the opening 140. In such embodiments, the cassette 300 may include a ramp 322 (see also FIGS. 3A-3C) to shift the IOL 200 upwards toward the opening 140 as the plunger 400 pushes the IOL into the nozzle 112. In some embodiments, the injector device 100 may include a port (not shown) through which saline or other liquids may be injected into a cavity 104 to provide lubrication.

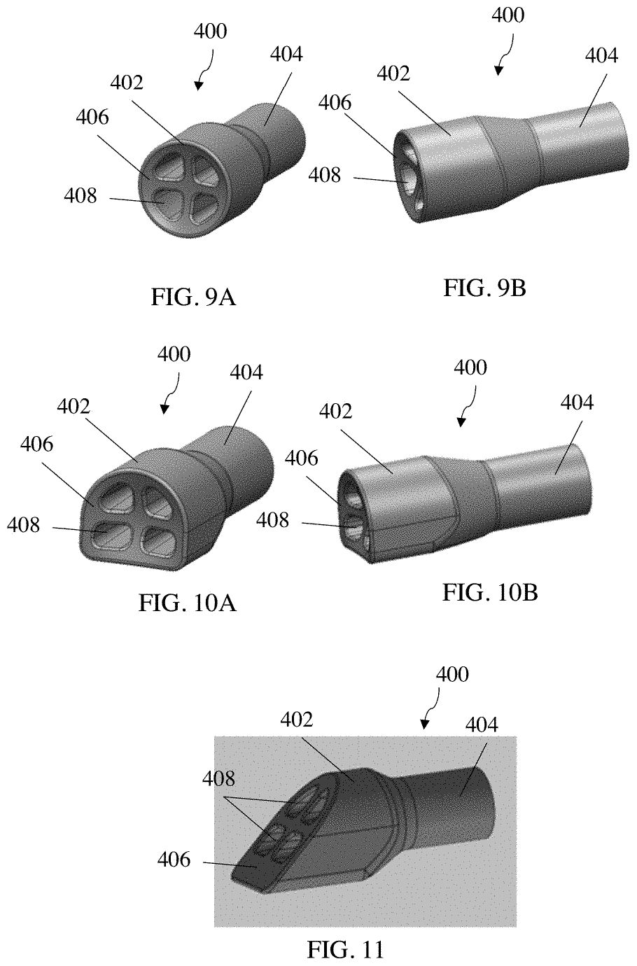

FIGS. 9A-9B, 10A-10B, and 11 show various embodiments of a plunger 400. As shown in the figures, a plunger 400 may include a distal portion 402 and a proximal portion 404 operatively coupled to a deployment system (not shown). In some embodiments, the distal portion 402 includes a distally oriented surface 406 that is configured to contact an IOL. The distally oriented surface 406 may include one or more openings 408 that extend from the distally oriented surface into the plunger. The openings 408 may permit the plunger to be radially compressed as the plunger is displaced from the cavity to the narrow diameter of the nozzle. The distal portion 402 may also be made of a compressible material to reduce the force for moving the plunger through the narrowing nozzle.

In some embodiments, shown in FIGS. 9A-9B, the distal portion 402 of the plunger may have a round shape. The distally oriented surface 406 may have a concave surface to complement the shape of the haptics as the IOL is maintained in the cavity. In some embodiments, shown in FIGS. 10A-10B, the distal portion 402 plunger may include a rounded portion and a flat portion. This may be more advantageous in some applications as the flat portion may glide more easily along a top surface of the wall 126. The distally oriented surface 406 may also be concave to complement the curve of the haptics, and the bottom corners of the distally oriented surface may contact distal ends of the haptics, helping to push the IOL distally.

As shown in FIG. 11, in some embodiments, the distal portion of a plunger may be rounded with a flat bottom, similar to the embodiment of FIGS. 10A-10B, but the distally oriented surface 406 may be a flat inclined surface such that the distal portion 402 resembles a wedge shape. Appropriate angles for the inclined distal surface of the plunger may be between or equal to 30 degrees and 60 degrees, 40 degrees and 50 degrees, and/or any other appropriate angle. During actuation, a bottom portion of the distally oriented surface 406 may contact a proximal most portion of a haptic of an IOL. This may help to push the IOL through the nozzle and the last haptic out of the distal opening without pushing as much material (i.e., of a plunger) through the nozzle, thus reducing the amount of force needed to eject the IOL from the injector device.

It should be noted that although specific shapes for a plunger are described above, other designs are contemplated as the disclosure is not limited to any particular plunger construction.

It should also be noted that a cassette may be inserted into a cavity of the injector device other than by rotational motion of a cover. For example, as shown in FIG. 14, a cassette 300 may be directly inserted into cavity 104 of the injector device 100 by moving the cassette in a downward motion into the cavity in a direction approximately normal to an opening in a body of the injection device. The cassette 300 may include tabs 211 on a bottom portion that may snap into corresponding indents in the cavity to position and secure the cassette into the cavity 104. However, other appropriate connectors and attachments methods may also be used as the disclosure is not so limited.

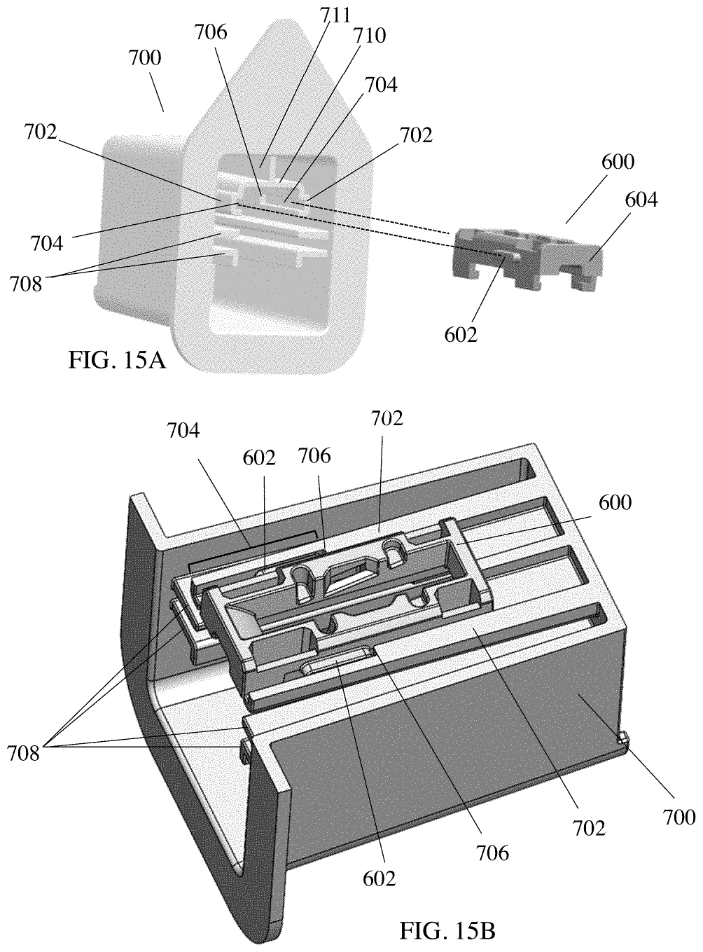

FIGS. 15A-15B illustrate an embodiment of a cassette 600 and a sterile package 700 for storing the cassette prior to use. Although not shown in FIGS. 15A-15B, an IOL may be stored in the cassette 600 in a nondeformed configuration (see FIG. 4A). With the cassette 600 positioned in the package 700, a surgeon or other user may insert a portion of the intraocular injection device into the package to load the cassette 600 onto the device. In some embodiments the package 700 includes opposing rails 702 that extend longitudinally from an opening of the package into an interior, and in some instances, to a back portion of the package opposite the opening. The rails 702 may be connected by a top portion 710 extending therebetween, and the top portion may be supported by a longitudinal rib 711 extending between an adjacent interior surface of the package 700 and the top portion 710. The rails 702 and top portion 710 may be shaped and sized to conform to at least a corresponding portion of a cross-sectional shape of the cassette 600 such that the cassette may be slid longitudinally into and supported within the package 700 via the rails 702.

In some embodiments, the cassette 600 may include protrusions 602 arranged on opposing sides of the cassette 600 configured to slide into cutouts 704 formed on inner surfaces of the rails 702 to hold the cassette in the package. In some instances, the protrusions 602 may be positioned closer to a first end 604 of the cassette 600 to assist a user in properly inserting the cassette 600 into the package 700. FIG. 15B is a cross-sectional schematic of the cassette 600 disposed within the package 700. The cutouts 704 may be configured such that when the cassette 600 is inserted into the package in the proper position, the protrusions 602 abut surfaces 706 of the cutouts 704, as shown in FIG. 15B. In this configuration, the cassette 600 is disposed entirely within an interior volume of the package 700. If a manufacturer or other user assembling the package tried to insert the cassette 600 into the package 700 in the wrong direction (e.g., inserting end 604 first) then the protrusions 602 would abut surfaces 706 in the different orientation in a manner that causes the cassette to stick out from the package opening due to the protrusions being offset from a middle of the cassette relative to a longitudinal length of the cassette and insertion direction into the package.

In some embodiments, a user may load the cassette 600 directly onto the intraocular injection device by inserting a cover 106 of the device into the package in a manner similar to that described above with respect to FIGS. 12A-12B. In some embodiments, the opening of the package 700 may include two pairs of parallel ledges 708 on opposite walls to receive outside edges of the cover 106. The ledges 708 which may be configured to aid in properly aligning the cover with the cassette 200 as the cover 106 is inserted into the opening of the package in a longitudinal direction.

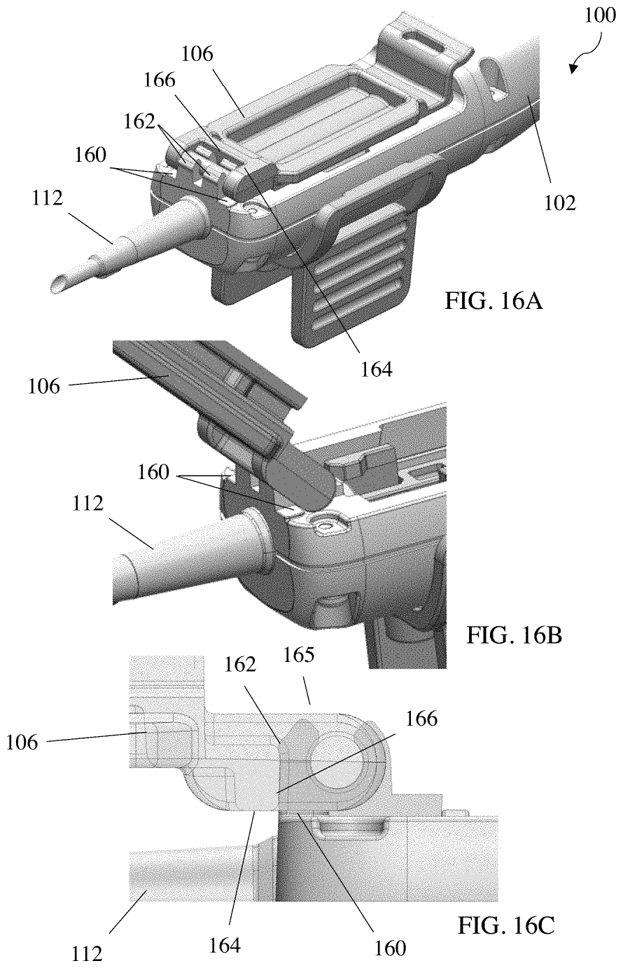

In some instances it may be desirable to maintain a desired stable orientation of a cover during engagement with a cassette. Accordingly, in some embodiments, as shown in FIGS. 16A-16C, an intraocular injector device 100 may include one or more features configured to support the cover in a desired open configuration. This may include, for example, bump outs 160 and/or ridges 162 that are configured to stabilize and maintain a cover 106 in a desired open configuration with a predetermined orientation. In the open configuration, a user may attach a cassette to the cover, e.g., by inserting the cover into a cassette package to load the cassette. As shown in FIGS. 16A-16C, in some embodiments, bump outs 160 may correspond to one or more protrusions that extend outward from an outer surface (e.g., a top surface) of the body 102 towards an opposing surface of the cover 106 when the cover is in the fully open configuration. In some instances, the bump outs may be arranged on opposing sides of a cover hinge 165. The bump outs 160 may have smooth surface area that abuts a surface 164 of the cover 106 when the cover is in the open configuration. For example, the bump outs may also include a surface that is configured to be disposed against a correspondingly shaped surface (e.g., both surfaces may be flat or otherwise have complimentary shapes) of the cover in some embodiments. The bump outs 160 may be sized and shaped to help maintain the cover 106 in a desired orientation to aid in properly aligning the cover for cassette attachment and prevent the cover from over-extending past a desired orientation. For example, the bump outs 160 may have a flat surface that is configured to maintain the cover 106 in an orientation that is approximately parallel to a longitudinal axis of the device, as shown in FIG. 16C. However, the bump outs 160 may also be configured to hold the cover 106 at any other angle suitable for attaching a cassette to the cover 106 as the disclosure is not limited in this fashion.

In some embodiments, the cover hinge 165 may include one or more detents 162 on a distal side of the hinge configured to engage with a corresponding feature on an inner surface 166 of the cover 106 adjacent the hinge. The detents may selectively lock the cover 106 in the fully open configuration such that a force greater than an unlocking force associated with the one or more detents, or other lock, may be applied to disengage the cover 106 from the detents and move the cover from the open configuration towards the closed configuration. As such, the one or more detents, or other appropriate lock (e.g., a latch, magnetic lock, etc.), may be used to help maintain the cover 106 in the open configuration to facilitate loading a cassette onto the cover 106.

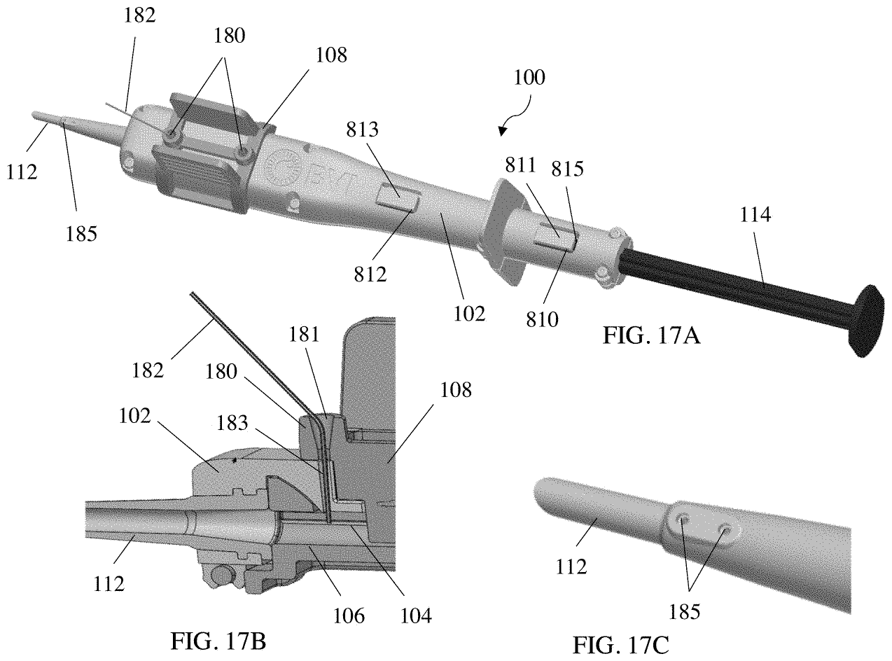

In some embodiments, a surgeon or other user may inject a fluid such as an ophthalmic viscoelastic device (“OVD”) or other viscous fluid into a cavity 104 in which an IOL is held prior to injection to reduce risk of scratching the IOL during deployment and/or to help provide lubrication during loading and/or deployment. In some embodiments, as shown in FIGS. 17A-17B, a shuttle 108 may include one or more bosses 180 with passages 181 extending through the bosses, and/or other appropriate portion of the body, for inserting a cannula 182. The body 102 of the injection device 100 may include passages 183 that extend from an external surface of the body 102 into the cavity 104 and positioned to connect to passages 181 of the shuttle when the shuttle is attached to the body 102 such that a flow path extends through the shuttle and body into the internal cavity of the body. As such, a user may inject an OVD into the cavity through passages 181, 183 via a cannula 182 or other appropriate OVD injection device. In some embodiments, the cannula may be made of stainless steel.

In some embodiments, it may be desirable to provide OVD to multiple portions of the cavity 104 of a system. Thus, the shuttle 108 may include both a distal and a proximal boss 180 providing two access passages to inject an OVD into the cavity 104 (e.g., proximal and distal relative to a loaded IOL in the device). In some embodiments, the bosses 180 may extend a distance outwards from a surface of the body 102 to prevent a cannula 182 from being inserted too far into the cavity 104. The cannula 182 may also be shaped with a curvature to prevent excessive insertion, as shown in FIG. 17B. In some embodiments, an opening of the passages 181 on the shuttle may be tapered outward toward the opening to allow for easy insertion of the cannula 182.

During operation, a user may insert the OVD through the one or more bosses 180 into the cavity 104 of the system prior to deploying a loaded IOL. As described above, the shuttle 108 may include a projection 110 (see FIGS. 5C and 6A) that maintains a position of a loaded IOL while injecting the OVD. In some embodiments, the bosses 180 are positioned on an upright surface of the injector device when the shuttle 108 is attached to the body 102 of the device. As such, a user may hold the device 100 in the upright position while injecting the OVD through bosses 180. The user may then detach the shuttle 108 and deploy the IOL from the device 100 which is already in the proper upright position. In some embodiments, as shown in FIGS. 17A and 17C, a nozzle 112 may include one or more venting ports 185 that are in fluid communication with the cannula, or other internal channel, extending through the nozzle that the IOL is deployed through. During deployment of the IOL, excess OVD may vent out of the venting ports 185. The venting ports may also reduce air pressure within the cannula of the nozzle which may reduce a risk of injecting air bubbles into the lens sack of a patient.

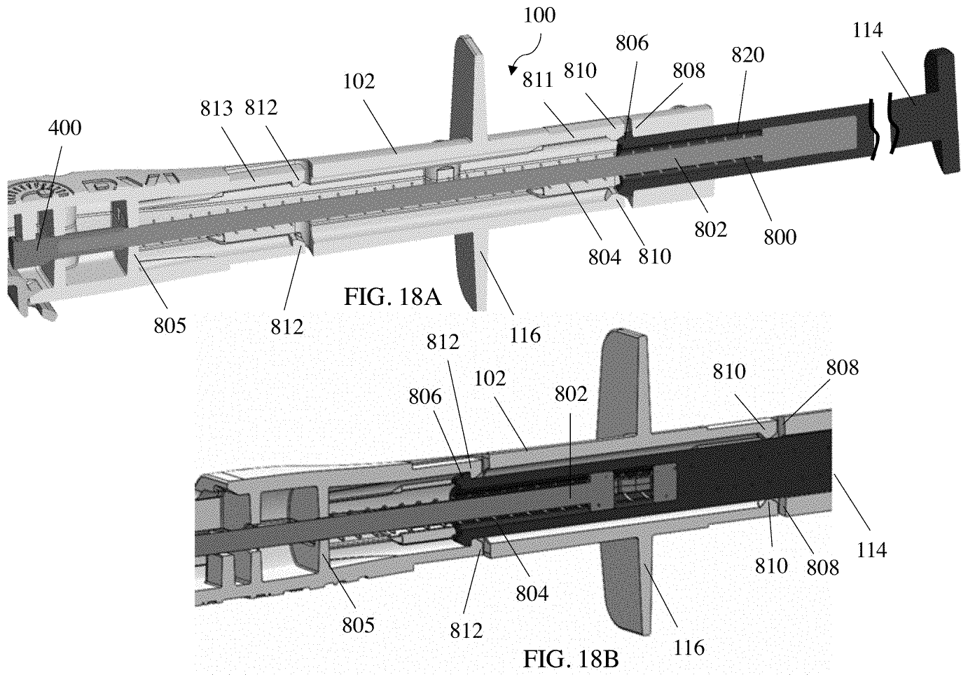

FIGS. 18A and 18B illustrate cross-sectional schematics of an intraocular injector device 100 prior to deployment and mid-way through deployment, respectively. In some embodiments, the device includes a piston 114 at a proximal end portion for actuating a plunger 400 to deploy an IOL out of a nozzle at a distal end portion. In some embodiments, the plunger 114 may include a spring-loaded inset portion 802 to lengthen a distance between the piston 114 and the plunger 400. The inset portion 802 may be concentrically positioned with the piston and may extend a distance into a distal cavity 800 of the piston 114. A compression spring 804 may be arranged around inset portion 802 between a proximal portion of the inset portion and a wall 805 arranged in the device body 102 distal to the piston 114 and proximal to a position of the plunger 400 prior to deployment.

In some embodiments, a distal end of the piston 114 includes radially outwardly extending protrusions 806 configured to contact first and second detents 810, 812 arranged along internal surfaces of the device body 102 at different lengths (see also FIG. 17A). Spring 804 may provide a small resistance force to movement of the plunger which may help avoid inadvertent actuation of the plunger 400. Thus, the detents may selectively prevent movement of the plunger in one or more directions until a threshold force is applied to the plunger. In some embodiments, first detents 810 are positioned near a proximal end portion of the body 102 and are configured to engage protrusions 806 of the piston 114 to hold the piston prior to deployment. In some embodiments, first detents 801 may be arranged at end portions of a lever 811 on the body 102 of the device to allow the detents 810 to flex radially outward when protrusions 806 are pushed distally past the detents. Protrusions 806 and first detents 810 may have correspondingly tapered surfaces that act as a cam to push the detents 810 radially outward as the protrusions 806 pass by. In some embodiments, ledges 808 in the device body located proximal to detents 810 may secure protrusions 806 to prevent piston 114 from being inadvertently pulled out distally from the device.

In some embodiments, the body 102 includes second detents 812 arranged proximal to the first detents 810. Second detents 812 may be arranged at end portions of a lever 813 on the body 102 of the device to allow the detents 812 to flex radially outward when protrusions 806 are pushed distally past the detents 812. Second detents 812 may have tapered surfaces similar to tapered surfaces of protrusions 806, though other appropriate detent constructions for either set of detents may be used as the disclosure is not so limited. The tapered surface may act as a cam to push the detents 812 radially outward as the protrusions 806 pass by until the protrusions engage distal surfaces of the detents 812 in a mid-deployment position. The second detents 812 may be positioned along a length of the device body 102 such that when the piston 114 engages in the mid-deployment position, the plunger 400 may have been actuated to push the IOL out of cavity 104 into nozzle 112.

FIG. 18B illustrates the piston 114 in the mid-deployment position. In the mid-deployment position, distal surfaces of the detents 812 may be engaged with protrusions 806 to prevent the piston 114 and plunger 400 from moving in a proximal direction. This may prevent the OVD and IOL being suctioned in a proximal direction back into the cavity 104 due to such movement which would result in a user needing to reposition the IOL. In some embodiments, a user may be able to pull the piston 114 proximally and disengage the second detents 812 with sufficient force if the user would like to return the piston to the first position. In some embodiments, in the mid-deployment position, only a force of the spring 802 needs to be overcome to move the piston 114 in a distal direction to actuate the plunger 400 and deploy the IOL. In some applications, a surgeon or other user may rest the device in the mid-deployment position to allow the IOL to relax and conform to an inner diameter of the nozzle 112 before final deployment. However, some users may prefer to not to stop at the second detents 812 and deploy the IOL by pressing the piston directly from the first position to final deployment in using a single motion.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. An intraocular lens injector device comprising:
a body including a cavity;
a projection extending into an interior of the cavity, wherein the cavity is configured to receive a cassette including an intraocular lens disposed therein, wherein when the cassette is inserted into the cavity, the projection deforms the intraocular lens to a predetermined configuration.

2. The intraocular lens injector device of claim 1, further comprising the cassette disposed in the cavity.

3. The intraocular lens injector device of claim 1, wherein the predetermined configuration is a curved shape.

4. The intraocular lens injector device of claim 1, further comprising a nozzle connected to the body and operatively associated with the cavity.

5. The intraocular lens injector device of claim 4, further comprising a plunger configured to move the intraocular lens through cavity and the nozzle.

6. The intraocular lens injector device of claim 5, wherein the nozzle is configured to deform to the intraocular lens a second predetermined configuration as the plunger moves the intraocular lens through the nozzle.

7. The intraocular lens injector device of claim 5, wherein the cassette includes an angled ramp at one end of the cassette configured to lift the intraocular lens from the cavity into an opening to the nozzle as the plunger pushes the intraocular lens into the nozzle.

8. The intraocular lens injector device of claim 5, wherein the body includes a first detent configured to selectively prevent the plunger from moving in a distal direction.

9. The intraocular lens injector device of claim 8, wherein the body includes a second detent configured to selectively prevent the plunger from moving in a proximal direction.

10. The intraocular lens injector device of claim 1, wherein the projection is configured to be selectively moved in a direction oriented away from the cavity to reduce a portion of the projection extending into the interior of the cavity.

11. The intraocular lens injector device of claim 10, wherein the projection is selectively removable from the body.

12. The intraocular lens injector device of claim 1, further comprising a cover moveably coupled to the body, wherein the cover is configured to be selectively moved between an open configuration and a closed configuration, and wherein the cover is configured to selectively couple the cassette thereto such that the cassette is disposed within the cavity when the cover is in the closed configuration.

13. The intraocular lens injector device of claim 12, wherein the cover is moved between the open and closed configurations via rotational movement.

14. The intraocular lens injector device of claim 12, further comprising one or more protrusions that extend outward from an outer surface of the body towards an opposing surface of the cover when the cover is in the open configuration, and wherein the one or more protrusions are configured to support the cover in the open configuration.

15. The intraocular lens injector device of claim 12, further comprising one or more detents configured to maintain the cover in the open configuration.

16. The intraocular lens injector device of claim 1, wherein the projection is configured to extend through an opening in the cassette to deform the intraocular lens.

17. The intraocular lens injector device of claim 1, wherein the projection includes a cutout configured to avoid contact with an optical portion of the intraocular lens during deformation of the intraocular lens.

18. A method comprising:
inserting a cassette into a cavity of an intraocular lens injector device, wherein the cassette includes an intraocular lens disposed therein; and
deforming the intraocular lens to a predetermined configuration with a projection extending into an interior of the cavity as the cassette is inserted into the cavity.

19-27. (canceled)

28. A cassette for holding an intraocular lens comprising:
a body configured to be inserted into a cavity of an intraocular lens injector device;
an opening extending through at least a portion of the body from a first surface of the body to a second surface of the body located opposite from the first surface, wherein the opening is configured to receive the intraocular lens therein; and
one or more ledges configured to support the intraocular lens in the opening of the cassette when the intraocular lens is disposed therein.

29. The cassette of claim 28, wherein the body is configured to be coupled to a surface of the intraocular lens injector device.

30. The cassette of claim 29, wherein the body is configured to be coupled to a movable cover of the intraocular lens injector device.

31. The cassette of claim 30, wherein the body includes one or more connectors configured to be attached to a rail on the cover.

32. The cassette of claim 31, further comprising a mechanical stop configured to prevent the cassette from sliding on the rail when the cassette is fully engaged with the cover.

33. The cassette of claim 28, wherein the one or more ledges are angled relative to the first surface.

34. The cassette of claim 28, further comprising an angled ramp that extends distally from the first surface towards the second surface along a deployment path of the intraocular lens from the cassette.

35. The cassette of claim 28, further comprising one or more haptic recesses extending into the first surface configured to receive one or more haptics coupled to the intraocular lens.

36. The cassette of claim 35, wherein the cassette includes one or more haptic recesses configured to receive one or more corresponding haptics of the intraocular lens.

37. The cassette of claim 35, wherein the one or more haptic recesses includes two or more haptic recesses, and wherein the two or more haptic recesses are located at different depths relative to the first surface.

38. The cassette of claim 28, further comprising a detent configured to hold the intraocular lens on the one or more ledges.

39. The cassette of claim 28, further comprising the intraocular lens disposed in the opening.
